(19) United States
(12) Patent Application Publication
(10) Pub. No.: US 2025/0160700 A1
(43) Pub. Date: May 22, 2025

(54) METHODS AND SYSTEMS FOR ESTIMATING BLOOD ANALYTE CONDITIONS

(71) Applicant: JRE Star Investment Holdings, LLC, Farmington, UT (US)

(72) Inventors: Tyler Jamison Jarvis, Provo, UT (US); Andrew Isaac Williams, Provo, UT (US); Robert C. Davis, Provo, UT (US)

(21) Appl. No.: 18/955,868

(22) Filed: Nov. 21, 2024

Related U.S. Application Data
(60) Provisional application No. 63/601,494, filed on Nov. 21, 2023.

(51) Int. Cl. A61B 5/1455; A61B 5/00; A61B 5/145

(52) U.S. Cl. CPC A61B 5/14552; A61B 5/14532; A61B 5/7275; A61B 2562/0238; A61B 2562/0242

(57) ABSTRACT

Methods and systems for estimating blood analyte conditions. In some methods, signal data may be received from a non-invasive blood monitor. The signal data may then be processed to remove a mean offset component from the signal data. The processed signal data may be transformed to a frequency domain. A DC offset component may then be extracted from the transformed signal data, after which the remaining data, which may comprise data from which the extracted data was taken, may be used to predict a blood analyte condition.

### CROSS-REFERENCE TO RELATED APPLICATIONS

This application claims the benefit under 35 U.S.C. § 119 (e) of U.S. Provisional Patent Application No. 63/601,494, which was filed Nov. 21, 2023, and titled “Integrated Path Approximation: A Novel Approach to Modeling Macroscopic Photon Absorption in Highly Scattering Media,” which is hereby incorporated herein by reference in its entirety.

### SUMMARY

Noninvasive blood monitoring is a decades-old research problem that has proved to be frustratingly elusive. Data generated from a state-of-the-art infrared spectrometer, combined with new techniques to model physiological interactions, may reveal previously inaccessible signals. Disclosed herein are methods, which may be incorporated into various systems, for deriving distributional estimates for photon scattering and absorption behavior, which lead to simple closed-form models that accurately model photon scattering. These analytic models improve reliable physiological signal extraction and continuous noninvasive measurement of biomarkers of interest.

To reliably determine small signals from noisy data, reasonable models of the underlying signal-generating process must be developed. This has proved to be difficult in the case of, for example, glucose measurement, notably because the light-particle interactions present are difficult to model in a closed-form way. Monte Carlo methods exist, but such Monte Carlo methods would be intractable for real-time measurements on embedded hardware.

Disclosed herein are closed-form models that reasonably approximate light-particle interactions in the body, and may therefore facilitate data assimilation efforts, along with estimation and/or prediction of various conditions, such as blood analyte conditions. Methods for extracting DC offset data from a data set are also disclosed herein. In some embodiments and implementations, such offset data may be extracted from a data set comprising a pulsatile signal and a non-pulsatile blood component. The DC offset component in such cases may comprise data derived from tissue, which may be extraneous for purposes of analyzing analytes in the blood. Such extracted data may therefore, in some cases, be discarded. Extracting such data may therefore be important in enhancing the accuracy of data processing techniques intended to estimate, predict, and/or correlate data with blood analyte conditions.

Techniques for removing and/or extracting certain components from a pulsatile signal used for estimating certain conditions, such as blood analyte conditions, are also disclosed. For example, because the DC offset component of a non-invasive electromagnetic radiation based sensor system typically contains data that may not be useful, and may make it more difficult to obtain useful information from the data, techniques disclosed herein may be used to extract the DC offset component, which in the case of a non-invasive blood monitoring system may leave only data associated with the pulsatile signal (e.g., the heartbeat being transduced through a blood vessel wall), along with data corresponding to the non-pulsatile blood component of the signal (i.e., electromagnetic waves reflected from the blood itself).

In an example of a method for processing signals from a non-invasive blood analyte tracking system, the method may comprise receiving signal data from a non-invasive blood monitor. The signal data may comprise a frequency component corresponding to a heart rate pulse of a user of the non-invasive blood monitor; a non-pulsatile blood component corresponding to light reflected from within a blood vessel; and a tissue-dependent DC offset component. The signal data may be processed to extract one or more components, such as the tissue-dependent DC offset component. The processed signal data may then be evaluated to provide an estimate of a blood analyte condition using only one or more of the remaining components, such as the frequency component and the non-pulsative blood component, from the signal data.

In some implementations, the blood analyte condition may comprise a concentration of the blood analyte.

In some implementations, the blood analyte condition may comprise a trend over time associated with the blood analyte.

In some implementations, the blood analyte may comprise glucose. Alternatively, or additionally, the blood analyte may comprise oxygen.

In some implementations, the step of processing the signal data may comprise use of a probability density model, such as a Levy Distribution.

In an example of a method for extracting a DC offset component from signal data using a non-invasive blood monitor according to some implementations, the method may comprise receiving signal data from a non-invasive blood monitor and processing the signal data to remove a mean offset component from the signal data. The processed signal data may be transformed to a frequency domain. A DC offset component may then be extracted from the transformed signal data. The remaining data, such as pulsatile signal data and/or non-pulsatile signal data from the blood, may then be used to predict a blood analyte condition.

In some implementations, the blood analyte condition may comprise a concentration of the analyte and/or a trend over time associated with the analyte.

The blood analyte may comprise any desired constituent of blood, such as glucose or oxygen, for example.

Some implementations may further comprise adjusting signal data to reduce scale discrepancies.

Some implementations may further comprise processing the signal data to compute discrepancy measure.

Some implementations may further comprise adjusting the offset to reduce discrepancy.

In an example of a method for estimating a blood analyte condition using data from a non-invasive blood monitor according to some implementations, the method may comprise receiving signal data from a non-invasive blood monitor. The signal data may comprise various separable constituents. For example, in some cases, the signal data may comprise a frequency component corresponding to a pulsatile signal, such as a heart rate pulse, of a user of the non-invasive blood monitor; a non-pulsatile blood component corresponding to light reflected from within a blood vessel; and a tissue-dependent DC offset component. One or more of these components may then be extracted. For example, some implementations may comprise extracting the non-pulsatile blood component and/or the DC offset component from the signal data. The extracted component(s) may then be used to estimate a blood analyte condition, such as a blood glucose level or blood oxygen level, for example.

Some implementations may further comprise extracting the frequency component. In some such cases, the frequency component may be used with the non-pulsatile blood component to estimate the blood analyte condition.

The blood analyte condition may comprise, for example, a trend over time in a concentration of the blood analyte and/or a concentration of the blood analyte.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a diagram illustrating a, arrangement for a standard Beer-Lambert law experiment;

FIG. 2 is a diagram illustrating a single scattering event in which o is the change in rotational angle from the old path, and θ is the change in azimuth angle from the old path;

FIGS. 3A and 3B are graphs comparing the Von Mises Distribution with the wrapped normal distribution;

FIGS. 4A and 4B are graphs comparing the density and cumulative density of the Von Mises vs. Uniform Distributions;

FIGS. 5A and 5B are graphs illustrating True CDF vs. Arctan Estimate of HG function, along with samples generated using inversion sampling;

FIG. 6A is a graph illustrating the Hoeffding dependence across scattering probabilities;

FIG. 6B is a graph illustrating the density of samples taken;

FIG. 7 is a graph illustrating an empirical CDF of samples generated from Monte-Carlo simulation of photon update equations according to some implementations compared to the modified Levy distribution;

FIG. 8 is a diagram illustrating a framework for a model involving photon paths from the surface of a user's skin to an artery underneath the skin;

FIG. 9A is a graph plotting experimental data using the loss function and plotting the loss for various values of x and y corresponding to various DC offset values;

FIG. 9B is another graph plotting synthetic data generated using the techniques of certain implementations described herein;

FIG. 10A is a graph schematically illustrating the constituent components of a signal having a pulsatile portion, a non-pulsatile blood component, and a DC offset/tissue component;

FIG. 10B is a diagram illustrating the various electromagnetic waves that correspond to the signal components illustrated in FIG. 10A;

FIG. 11 is a flowchart illustrating a method for extracting a DC offset component of a signal according to some implementations;

FIG. 12 is a block diagram illustrating an example of a system for extracting one or more components from a pulsatile signal; and

FIG. 13 is a flowchart illustrating an example of a method for estimating a blood analyte condition according to some implementations.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail.

As used herein, the term “substantially” refers to the complete or nearly complete extent or degree of an action, characteristic, property, state, structure, item, or result to function as indicated. For example, an object that is “substantially” cylindrical or “substantially” perpendicular would mean that the object/feature is either cylindrical/perpendicular or nearly cylindrical/perpendicular so as to result in the same or nearly the same function. The exact allowable degree of deviation provided by this term may depend on the specific context. The use of “substantially” is equally applicable when used in a negative connotation to refer to the complete or near complete lack of an action, characteristic, property, state, structure, item, or result. For example, structure which is “substantially free of” a bottom would either completely lack a bottom or so nearly completely lack a bottom that the effect would be effectively the same as if it completely lacked a bottom.

Similarly, as used herein, the term “about” is used to provide flexibility to a numerical range endpoint by providing that a given value may be “a little above” or “a little below” the endpoint while still accomplishing the function associated with the range.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

A classical way to understand light-particle interactions is the Beer-Lambert law, which states that the macroscopic behavior of light when a narrow-beam light source propagates through a diffusive media is governed by the following equation:

$$I = {I}_{0} {e}^{- ε lc}$$

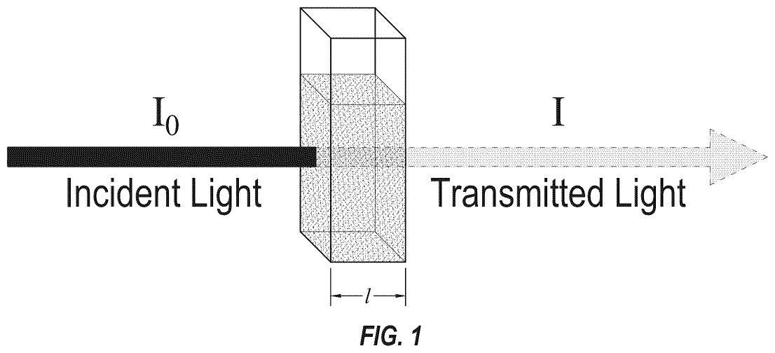

FIG. 1 illustrates conceptually the framework for the standard Beer-Lambert law experiment and equation, in which I is the intensity of detected/transmitted light, I₀ is the initial intensity of the light, ε is the molar absorption coefficient (material dependent), l is the optical path length, and c is the concentration of the absorptive species in the media. Notably, this equation only applies when the path length is constant for all detected paths (or can be approximated to be constant) and the media has a uniform distribution of absorbing media.

In the case of a body-spectrometer system, however, such approximations do not reflect the physical reality of the system. Other work related to physiological signal extraction includes the differential Beer-Lambert law, useful in continuous-wave near-infrared tissue spectroscopy (cwNIRS) for measuring changes in tissue chromophore concentrations, particularly oxy- and deoxyhemoglobin. The differential Beer-Lambert law is expressed as:

$$Δ A = ln \frac{{I}_{det , 1}}{{I}_{det , 2}} = L Δ {μ}_{a}$$

where ΔA represents the change in light attenuation, I_{det,1} and I_{det,2} are the detected light intensities in two different tissue states, L is the mean path length of detected photons, and Δμₐ is the change in the absorption coefficient.

This law posits that the change in light attenuation is directly proportional to changes in chromophore concentrations, allowing for the quantification of these changes based on detected light intensities. However, the differential Beer-Lambert law relies on two critical assumptions: (1) homogeneous absorption changes within the tissue, and (2) constant scattering loss. Deviations from these assumptions, such as inhomogeneous absorption changes and variations in scattering, can lead to underestimations in concentration changes (partial volume effect) and crosstalk between chromophores.

This disclosure provides a new model that is more expressive than the differential model, as it models not only the change in absorption but also absolute absorption values, thereby providing more information about the observed physiological system. Moreover, the model derived herein is more representative of the true physics of photon scattering in turbid media, which may therefore provide a suitable path for improving signal processing in blood and other body fluids.

Instead of considering all photons simultaneously, it may be useful to study the path of a single photon packet at a time. We can consider a small part of the path of this photon and may be able to make several assumptions. For example, we may assume that this section of path is small enough that the scattering media can be assumed to be uniformly distributed along the section. We may also be able to assume that this section of the path is large enough that we still observe the macroscopic effects of the Beer-Lambert law. Finally, we may assume that refraction due to Snell's law is negligible given the multitude of other scattering events.

Given these assumptions, we can model such a section of the path as:

$${I}_{t + 1} = {I}_{t} {e}^{- {ε}_{t} {lc}_{t}}$$

where cₜ and εₜ depend on time, and l is fixed. Since this is a recursive equation, we can write:

$${I}_{t + 1} = {I}_{0} {e}^{{\sum }_{j = 0}^{t} - {ε}_{j} {lc}_{j}}$$

Since each path is a random walk with an absorbing barrier (i.e., absorbed if the particle leaves the body or is detected), we may consider the final time Iₜ_{_{f}}. Since summation is linear, and by the property of n̅ the algebraic mean Σᵢ^{N}nᵢ=Nn̅, we can write the following:

$${I}_{{t}_{f}} = {I}_{0} {e}^{{\sum }_{j = 0}^{{t}_{f}} - {ε}_{j} {lc}_{j}} = {I}_{0} {e}^{- l {\sum }_{j}^{A} {ε}_{j} {c}_{j}} = {I}_{0} {e}^{- lA \overline{ε}}$$

where A is the number of steps the photon took before leaving the body or being detected, and ε̅ is the average absorption along the path. Note that c̅ₜ is combined with ε̅ₜ since they are indistinguishable in the sum. Thus, we have reduced the absorption along a path down to a Beer-Lambert like expression, but this only applies to a single photon path. In order to calculate the resultant detected light at a particular wavelength, we have the expression:

$${I}_{D} = \frac{{I}_{0}}{N} {\sum }_{j}^{N} {e}^{- {lA}_{j} {\overline{ε}}_{j}}$$

where I_{D} is the bulk detected light (we only sum over detected paths), I₀ is the total initial intensity of light, (thus

$$\frac{{I}_{0}}{N}$$

is the intensity of light per packet,), Aⱼ is the number of steps the photon packet took before being detected, and ε̅ⱼ is the average absorption of a single path.

It is worth noting the similarity of this expression to the Feynman-Kac formula, exchanging integrals for summation, as necessary. This is an interesting connection that bears more investigation. Now, if we wish to simplify this model further, we must understand Aⱼ and ε̅ⱼ.

We begin with ε̅ⱼ, as it is the easier of the two. We can consider each εⱼ as a sample from a categorical distribution since for each step along the path, we end up in one of M different body tissue compartments, with some probability p₁ . . . pₘ. Note that we may consider this categorical distribution to be fixed across one path, since the summation in the recursive approximation is linear, so we can rearrange tissue compartments as we like.

Proposition 1:

$${I}_{{t}_{f}} = {I}_{0} {e}^{- l {\sum }_{j}^{A} {ε}_{j} {c}_{j}} = {I}_{0} {e}^{- l {\sum }_{j}^{A} {\tilde{ε}}_{j} {c}_{j}}$$

where the \tilde{ε}ⱼ are i.i.d. categorical random variables defined only by Aε̅

Proof. Nε̅=Σᵢ₌₁^{N}εᵢ and we have that given K different possible values of ε, then since E[v]=Σᵢ₌₁^{K}pᵢvᵢ, we can define a categorical distribution to make it work.

This is because we have Nε̅=Σᵢ₌₁^{N}εᵢ, and since all we observe is the bulk absorption across the path, we can reorder the εᵢ as we like, thus we can take them as i.i.d. Thus, each εⱼ is a sample from some categorical distribution, with each εⱼ independent from the others. Thus the εⱼ are i.i.d. and so by the Central Limit Theorem (CLT), the ε̅ⱼ, have a normal distribution. Note this distribution is not necessarily the same distribution across different paths; in fact, we expect it to be different.

To understand the Aⱼ, we must embark on a journey of probability theory. We can first re-frame our definition of Aⱼ. We have that lAⱼ=L for some total path length L. If we instead consider small time steps over our photon path and assume the velocity of light is constant across the path (a reasonable approximation since the light is mainly propagating through water, no matter the tissue compartment), then we can write L=cAⱼ, where Aⱼ is now the time the photon has spent in the body, and c is the speed of light in the body. Given this assumption, we will henceforth refer to a path's length in terms of its time since the two are equivalent given constant velocity.

In preferred embodiments, an important feature of the model is the fact that the Aⱼ are distributed according to a modified Levy Distribution, which is the distribution of hitting times of Brownian motion. This section is dedicated to the proof of that fact. The density function for the hitting time of the A; will henceforth be denoted [symbol] (·|d, σ), defined as:

$$ℒ ( t \vert d , σ ) = \frac{d}{\sqrt{2 π {σ}^{2}}} {t}^{- 3 / 2} {e}^{{d}^{2} / ( 2 t {σ}^{2} )} ,$$

where σ is the dispersion coefficient (which is related to the variance of the random walk, or rather the proclivity of scattering), and d is the distance of the absorbing (detection) barrier from the starting point of the path. This distributional information will allow us to make simplifications that respect the underlying probability laws, which provides simple, closed-form approximations that are accurate and computationally efficient. Much of the work done here is to develop and justify these simplifications.

To prove that the density of the Aⱼ is given by the equation above, we can simplify the three-dimensional case to the one-dimensional case. We note that to understand when paths leave the skin, we need only consider the movement in the z-direction, where we consider the x and y directions to be coplanar with the surface of the skin. We also consider the skin to be a flat Cartesian plane, ignoring the curvature of the surface of the skin. For most purposes, this is a reasonable approximation since any photon detector is localized to a small area of the wrist, where curvature is typically negligible. Moreover, any photons that travel a significant distance from the incidence point of the photon source will have been mostly absorbed, so any contributions from these photons will be negligible.

Thus, to understand the distribution of lengths of paths, we may simply consider the length of time they spend in the tissue, which will be determined by the time they leave the surface of the skin. Then, the expression we must consider is only the z component of the random walk of the photons.

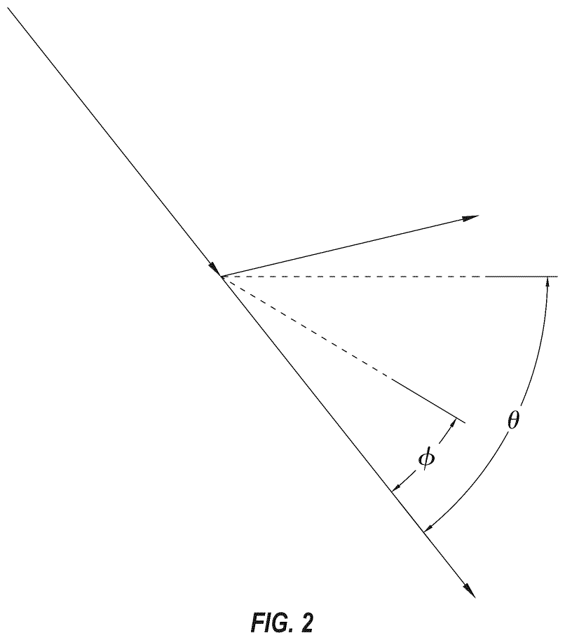

We can express the position of a photon using standard spherical coordinates, with x=rsin (θ) cos (ϕ), y=rsin (θ) sin (ϕ), z=rcos (θ). Then, scattering events will change the direction according to ϕ and θ. We may then consider such a scattering event as illustrated in FIG. 2, where θ is the change in inclination from the old trajectory, and ϕ is the change in rotation angle from the old trajectory.

A single scattering event illustrated in FIG. 2, in which ϕ is the change in rotational angle from the old path, and θ is the change in azimuth angle from the old path. The old path is illustrated with the dotted line, and the dashed lines illustrate the change in angle in the x,y and y,z planes, respectively.

Then, in order to consider the absolute position, we must add the update angles, since if a trajectory is moving at some angle θ₁ with respect to the skin, (or some fixed reference point) and θ₂ is the change in angle from the original trajectory, then the total angle, relative to the skin will be θ₁+θ₂, modulo 2π. Then, the absolute position of the photon at a given time step will be given by the following update equations:

$${x}_{n + 1} = {x}_{n} + l \cdot sin ( {\sum }_{i = 1}^{n} {θ}_{i} ) cos ( {\sum }_{i = 1}^{n} {ϕ}_{i} )$$

$${y}_{n + 1} = {x}_{n} + l \cdot sin ( {\sum }_{i = 1}^{n} {θ}_{i} ) sin ( {\sum }_{i = 1}^{n} {ϕ}_{i} )$$

$${z}_{n + 1} = {x}_{n} + l \cdot cos ( {\sum }_{i = 1}^{n} {θ}_{i} )$$

Then, we can model the movement in the z direction as a random walk, and we consider updates to the z position as drawn from some distribution. Photon scattering is often modeled using a Heyney-Greenstein distribution, denoted HG(·|g, α), which accounts for the anisotropy of scattering, parameterized by g, and the contribution of Rayleigh scattering, parameterized by α. This density function gives the probability of scattering at a certain angle θ away from the current trajectory of the photon. Then, for a typical photon scattering event, we will have the update angle drawn from the modified Henyney-Greenstein distribution, a standard photon scattering model in turbid media. Here we use a slightly modified distribution called the Draine distribution, which accounts for the effects of Rayleigh scattering, parameterized by α. We then define the density HG:

$$HG ( β \vert g , α ) = \frac{3}{4 π} \frac{1 - {g}^{2}}{3 + α ( 1 + 2 {g}^{2} )} \frac{1 + α {cos}^{2} ( β )}{{( 1 + {g}^{2} - 2 g cos ( β ) )}^{3 / 2}}$$

However, this will only account for one dimension of scattering. To calculate the updates to both θ and ϕ for a scattering event, we may assume that once the Heyney-Greenstein angle β is determined, the scattering direction will be uniformly distributed in the “cone” around the original trajectory, with vertex angle β. Thus, we will have that, for a given timestep, the updates will be given by θₙ=βₙ sin (uₙ) and ϕₙ=βₙ cos (uₙ), where uₙ is uniformly sampled over [0,2π].

Now, we note that scattering events will occur with some likelihood μₛ, such that over a fixed distance, a photon will have some probability μₛ of scattering or not. Thus, if we wish to update the absolute orientation of the photon, we will update the positions by multiplying on by a random variable sampled from a Bernoulli distribution with likelihood μₛ.

Thus, the absolute z position of the particle at time index n+1 will be given by:

$${z}_{n + 1} = l \cdot {\sum }_{i = 1}^{n} cos ( {\sum }_{j = 1}^{i} {τ}_{j} sin ( {u}_{j} ) {β}_{j} )$$

Where to τⱼ˜Bernoulli(μs), μⱼ˜Uniform (−π, π) and βⱼ˜HG(g, α).

The independence of scattering events can be modeled as follows:

$${z}_{n + 1} = l \cdot {\sum }_{i = 1}^{n} cos ( {\sum }_{j = 1}^{i} {τ}_{j} sin ( {u}_{j} ) {β}_{j} )$$

can be approximated to be independent from

$${z}_{n + M + 1} = l \cdot {\sum }_{i = 1}^{n + M} cos ( {\sum }_{j = 1}^{i} {τ}_{j} sin ( {u}_{j} ) {β}_{j} )$$

if M is sufficiently large, and so if

$${ζ}_{i} = l \cdot {\sum }_{i = 1}^{M} cos ( {\sum }_{j = 1}^{i} {τ}_{j} sin ( {u}_{j} ) {β}_{j} )$$

then the ζᵢ can be taken to be independent, and

$${z}_{nM} = {\sum }_{i = 1}^{n} {ζ}_{i}$$

is a random walk with the strong Markov property

Thus, to model this position as a random walk with the strong Markov property, we wish to find some step size M so that we can express the updates to the z position as independent, identically distributed (i.i.d.) random variables. This is justified since the τⱼ sin (uⱼ)βⱼ are i.i.d., and thus their sum will converge to a normal distribution by CLT. Then in order to get independence, we must pick M large enough so that after M steps, the positions become effectively independent, allowing us to encapsulate the M updates as a single random variable, which will be independent of the M+1 to 2M position updates. This independence fundamentally comes from the aliasing of the cos (x) function over its domain. Since cos (x)=cos (x+2π), then the aliasing of the domain “wraps” the distribution around the unit circle, a notion formalized with the wrapped normal distribution. The wrapped normal distribution has density function given by:

$${f}_{WN} = \frac{1}{σ \sqrt{2 π}} {\sum }_{k = - ∞}^{∞} exp ( \frac{- {( θ - μ + 2 π k )}^{2}}{2 {σ}^{2}} )$$

where μ, σ are the mean and variance of the unwrapped normal distribution. As o approaches infinity, the f_{WN} density approaches a Uniform distribution on [−π, π]. Then since the sum of the τⱼ sin (uⱼ)βⱼ will have a normal distribution with strictly increasing variance, then the aliasing of cos (x) will result in samples from cos (Σⱼ^{∞}τⱼ sin (uⱼ)βⱼ) approaching cos (Uniform(−π, π)).

Then to get independence, if we pick M large enough, we will have Σⱼ₌₀^{M}τⱼ sin (uⱼ)βⱼ normally distributed with some large variance. Then the wrapped distribution will be near to Uniform(−π, π), as will Σ_{j=M}^{2M}τⱼ sin (uⱼ)βⱼ. Then we can approximate these two sums as being independent since we will have for any yϵ[−1,1]:

$$P ( cos ( {\sum }_{j = 0}^{M} {τ}_{j} sin ( {u}_{j} ) {β}_{j} ) \leq y ) \approx P ( cos ( {\sum }_{j = 0}^{2 M} {τ}_{j} sin ( {u}_{j} ) {β}_{j} ) \leq y )$$

since the distribution of the sum of the τⱼ sin (uⱼ)βⱼ converges.

Lemma 1: The sum of two uniform random variables supported on the unit circle will be uniformly distributed.

Then, if we label:

$${η}_{1} = {\sum }_{j = 0}^{M} {τ}_{j} ( sin ( {u}_{j} ) {β}_{j} )$$

$${η}_{2} = {\sum }_{j = M}^{2 M} {τ}_{j} ( sin ( {u}_{j} ) {β}_{j} )$$

then we will have

$$P ( cos ( {η}_{1} + {η}_{2} ) \leq y ) \approx P ( cos ( {η}_{2} ) \leq y ) ,$$

which implies

$$\begin{matrix}P ( cos ( {η}_{1} ) \leq y , cos ( {η}_{1} + {η}_{2} ) \leq y ) \approx P ( cos ( {η}_{1} ) \leq y , cos ( {η}_{2} ) \leq y ) \\ \approx P ( cos ( {η}_{1} ) \leq y ) P ( cos ( {η}_{2} ) \leq y )\end{matrix} .$$

The second line follows from the fact that adding η₁ to η₂ essentially shifts the base point of the distribution of η₂. Thus, accounting for the aliasing of cos (x) over its domain has η₂ mod 2π distributed nearly uniformly, and so by the aforementioned Lemma, we have that their sum is also uniformly distributed.

Thus, knowing the value of cos (η₁) does not influence the value of cos (η₁+η₂), due to the density of η₂ mod 2π being uniformly constant. Therefore, we have that cos (η₁+η₂) and cos (η₂) are independent, and so we have that for some M

$$l \cdot {\sum }_{i = 1}^{M} cos ( {\sum }_{j = 1}^{i} {τ}_{j} sin ( {u}_{j} ) {β}_{j} )$$

is independent from

$$l \cdot {\sum }_{i = M}^{2 M} cos ( {\sum }_{j = 1}^{i} {τ}_{j} sin ( {u}_{j} ) {β}_{j} )$$

Thus, we can write:

$${z}_{M ( n + 1 )} = {z}_{M \cdot n} + {ζ}_{n}$$

Where the ζₙ are essentially i.i.d.

A Hoeffding test for independence of cos (η₁) and cos (η₁+η₂), with M given by the lower bound, we derive below. This test demonstrates empirical support for the validity of the assumptions of cos (η₁), cos (η₂) for sufficiently large M.

In order to continue our analysis on the ζₙ, we will want M to be as small as possible while still retaining independence. Thus, we derive a lower bound for M.

We need to work with the wrapped normal distribution to extract this lower bound. However, the wrapped normal distribution is difficult to work with, so we instead work with a good approximation to the wrapped normal distribution, the von Mises distribution. The wrapped normal distribution describes a freely diffusing angle θ on a circle with an unwrapped variance that grows linearly in time. On the other hand, the von Mises distribution is the stationary distribution of a drift and diffusion process on the circle in a harmonic potential, i.e., with a preferred orientation.

The density function for the von Mises distribution is given by:

$$f ( x \vert μ , κ ) = \frac{exp ( κ cos ( x - μ ) )}{2 π {I}_{0} ( κ )}$$

where l₀(κ) is the modified Bessel function of the first kind of order 0. Here, if a von Mises distribution is used to approximate a wrapped normal distribution, we have

$$κ = \frac{1}{{σ}^{2}} ,$$

where σ² is the variance parameter of the wrapped normal distribution, and μ is the same as in the wrapped normal distribution.

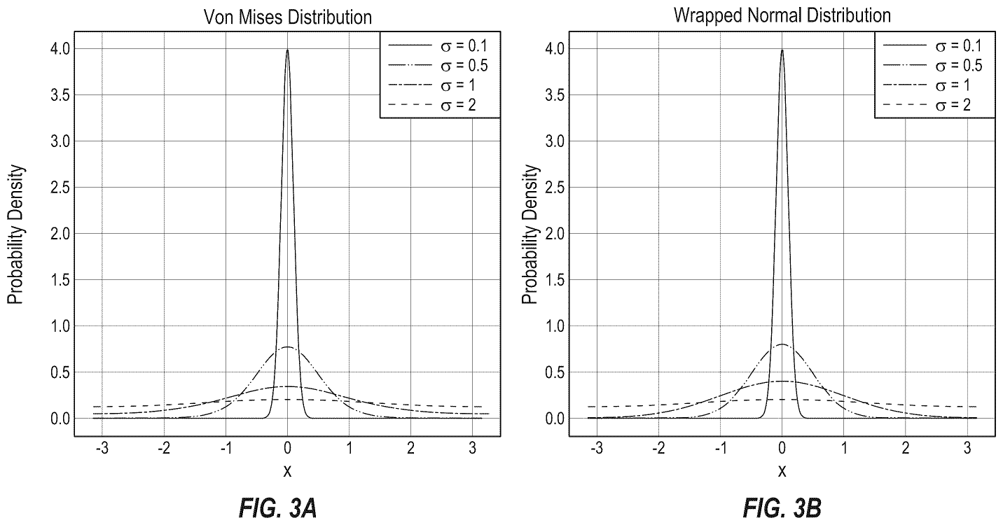

In FIGS. 3A and 3B, we show plots of the von Mises and wrapped normal distributions, respectively, to illustrate their similarity. It may be preferable to use the von Mises distribution because it is typically simpler to work with.

Then, in order to isolate a lower bound, we first set a target value κ for the von Mises distribution. if

$$κ = \frac{1}{{π}^{2}} ,$$

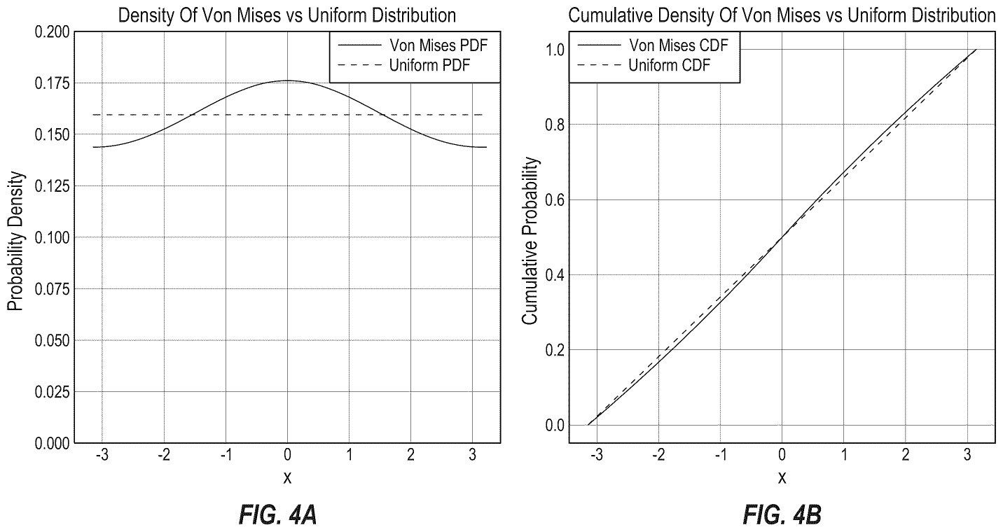

then the corresponding von Mises distribution is very nearly uniform. FIGS. 4A and 4B provide a comparison of the von Mises distribution with

$$κ = \frac{1}{{π}^{2}}$$

and uniform (−π, π). The Wasserstein distance between the two is 0.066.

This value for κ corresponds to a variance σ=π for the corresponding normal distribution. This value of σ heuristically comes from the fact that the derivative of the normal pdf will have a maximum when the second derivative is zero and a negative third derivative. We wish to characterize the maximal rate of change of the normal density function since we want to approximate a constant distribution once we wrap the normal distribution. Thus, by characterizing the maximal change, we can bound the uniformity of the wrapped distribution by the maximal rate of change of the unwrapped distribution.

The derivative of the normal pdf is given by:

$$\frac{d}{dx} {f}_{N} ( x \vert μ , σ ) = \frac{{xe}^{- \frac{{x}^{2}}{2 {σ}^{2}}}}{{σ}^{3} \sqrt{2 π}}$$

Where we have set μ=0 since the distribution of η is mean centered. Then, the derivative will be maximized when the second derivative:

$$\frac{{d}^{2}}{{dx}^{2}} {f}_{N} ( x \vert μ , σ ) = \frac{{e}^{- {x}^{2}} 2 {σ}^{2} ( {σ}^{2} - {x}^{2} )}{\sqrt{2 π} {σ}^{5}}$$

is zero, or when x=±σ. Then we have the third derivative:

$$\frac{{d}^{3}}{{dx}^{3}} {f}_{N} ( x \vert μ , σ ) = \frac{{e}^{\frac{- {x}^{2}}{2 {σ}^{2}}} ( - 3 {σ}^{2} x + {x}^{3} )}{\sqrt{2 {πσ}^{7}}}$$

is negative at x=±σ.

Then, since the aliasing of cos (x) will wrap the distribution onto [−π, π], we have if σ=π, then the maximal rate of change of the underlying distribution will be wrapped to the endpoints of the support of the wrapped distribution, and we will have some of this change offset by the wrapping at the point of maximal change. Then the corresponding von Mises distribution with

$$κ = \frac{1}{{π}^{2}}$$

is a reasonable approximation of the uniform distribution, and thus our independence results relying on the invariance of the final distribution of cos (η) to the aliasing split location go through.

Then we wish for Σⱼ^{M}τⱼ sin (uⱼ)βⱼ to be normally distributed with variance π². Thus, we analyze the variance of τⱼ sin (uⱼ)βⱼ in order to find a lower bound on M. We have that τⱼ is Bernoulli, which is discrete but can be represented by μₛδ(x−1)+(1−μₛ)δ(x), where δ(x) is the Dirac distribution. Then τⱼβⱼ will be distributed according to:

$${τ}_{j} {β}_{j} \sim {μ}_{s} HG ( x \vert g , α ) + ( 1 - {μ}_{s} ) δ ( x ) ,$$

since if τⱼ=1, then τⱼβⱼ˜HG(ϕ|g, α), and if τⱼ=0, then τⱼβⱼ=0. Then, since each of these events has probability μₛ and 1−μₛ, respectively, we simply weight each of the two cases by μₛ, and (1−μₛ). Then the final distribution will be the weighted sum due to the independence of τⱼ and βⱼ, so we have by the law of total probability:

$$\begin{matrix}P ( {τ}_{j} {β}_{j} \leq y ) = P ( {τ}_{j} {β}_{j} \leq y \vert {τ}_{j} = 1 ) P ( {τ}_{j} = 1 ) + \\ P ( {τ}_{j} {β}_{j} \leq y \vert {τ}_{j} = 0 ) P ( {τ}_{j} = 0 ) \\ = P ( {β}_{j} \leq y ) P ( {τ}_{j} = 1 ) + P ( 0 \leq y ) P ( {τ}_{j} = 0 ) \\ = {μ}_{s} {\int }_{- π}^{y} HG ( x \vert g , α ) dx + ( 1 - {μ}_{s} ) {\int }_{- π}^{y} δ ( x ) dx\end{matrix}$$

Thus, we have τⱼβⱼ˜μₛHG(x|g, α)+(1−μₛ)δ(x). Then the variance of τⱼβⱼ will be given by:

$$Var ( {τ}_{j} {β}_{j} ) = {\int }_{- π}^{π} {x}^{2} ( {μ}_{s} HG ( x \vert g , α ) + ( 1 - {μ}_{s} ) δ ( x ) ) = {μ}_{s} Var ( HG ( x \vert g , α ) ) + ( 1 - {μ}_{s} ) \cdot 0 = {μ}_{s} Var ( HG ( x \vert g , α ) ) ,$$

since the support of δ(x)={0}.

Then we wish to use the probability integral transform to get the distribution for sin (x). In order to use this transform, we need a bijective mapping so the inverse function is well-defined. Then, since sin (x) is an odd function, we can consider uⱼ distributed uniformly on [0, π], and then the distribution for the transform of a uniform random variable on [−π, π] will be an appropriately reflected and scaled version of the distribution for sin (uₙ). Note that sin (x) is not bijective on [0, π] but sin (x) is symmetric on this domain about π/2, and so we can simply consider sin (ûⱼ) for ûⱼ distributed uniformly on [0, π/2]. Then by the probability integral transform, that for ûⱼ˜Uniform(0, π/2):

$$P ( sin ( {\hat{u}}_{j} ) \leq y ) = P ( {\hat{u}}_{j} \leq arc sin ( y ) ) = \frac{2 arc sin ( y )}{π}$$

For yϵ[0,1]. Then we have by the fundamental theorem of calculus:

$$P ( sin ( {\hat{u}}_{j} ) \leq y ) = \frac{2 arcsin ( y )}{π} = {\int }_{0}^{y} \frac{d}{dx} \frac{2 arcsin ( x )}{π} d x = {\int }_{0}^{y} \frac{2}{π \sqrt{1 - {x}^{2}}} dx$$

Then, to get the distribution for sin (uⱼ) with uⱼ˜Uniform (−π, π), we reflect the distribution across the y axis and scale appropriately so the distribution integrates to unity. However,

$$\frac{2}{π \sqrt{1 - {x}^{2}}}$$

is symmetric about x=0, thus our density for sin (uⱼ) is given by:

$${f}_{sin ( {u}_{j} )} ( x ) = \frac{1}{π \sqrt{1 - {x}^{2}}}$$

for xϵ[−1,1]

Which has mean 0, (by symmetry), and variance given by:

$$Var ( sin ( {u}_{j} ) ) = {\int }_{- 1}^{1} \frac{{x}^{2}}{π \sqrt{1 - {x}^{2}}} = \frac{1}{2}$$

Then we have using the relation: Var(XY)=E(X²Y²)+E(XY)², that the variance of Var(sin (uⱼ)τⱼβⱼ) is given by:

$$Var ( sin ( {u}_{j} ) {τ}_{j} {β}_{j} ) = Var ( sin ( {u}_{j} ) ) Var ( {τ}_{j} {β}_{j} ) + Var ( sin ( {u}_{j} ) ) {E ( {τ}_{j} {β}_{j} )}^{2} + {E ( sin ( {u}_{j} ) )}^{2} Var ( {τ}_{j} {β}_{j} ) = \frac{1}{2} {μ}_{s} Var ( HG ( x \vert g , α ) ) + \frac{1}{2} \cdot 0 + 0 \cdot Var ( HG ( x \vert g , α ) ) = \frac{{μ}_{s}}{2} Var ( HG ( x \vert g , α ) )$$

Then the variance of the τⱼ sin (uⱼ)βⱼ is known. Then, for the purposes of variance computation, if we assume that the τⱼ sin (uⱼ)βⱼ are normally distributed, we have by the property of normal distributions N(0, σ₁²)+N(0, σ₂²)=N(0, σ₁²+σ₂²), that:

$${\sum }_{j = 0}^{M} {τ}_{j} sin ( {u}_{n} ) {β}_{j} ∼ N ( 0 , \frac{M {μ}_{s}}{2} Var ( HG ( x \vert g , α ) ) )$$

Thus, if we want a resulting distribution that has variance π², then we set:

$$M = \frac{2 {π}^{2}}{{μ}_{s} Var ( HG ( x \vert g , α ) )}$$

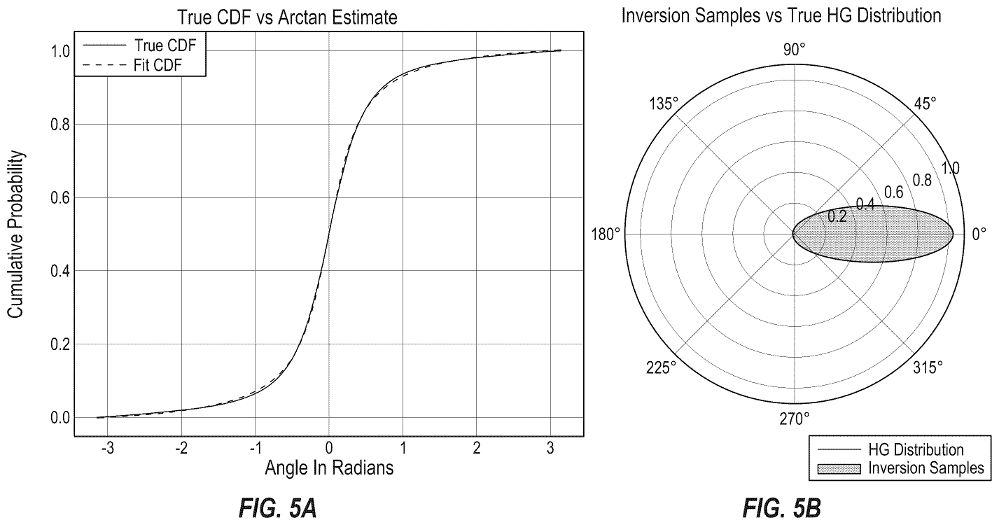

In order to sample from the distribution of ζₙ, we must sample the βₙ from the HG distribution. However, we wish to perform inversion sampling (so that samples can be generated quickly for other, more complex Monte-Carlo experiments), we must find an inverse to the CDF of HG(ψ|g, α). However, this CDF does not have an easily computable inverse thus, we use a parametric function of the form α+barctan (cx), and then given the CDF of an HG distribution (which we can compute analytically), we can then fit a least squares estimate of the CDF, and then invert the parametric equation to find F⁻¹ for HG(ψ|g, α). We illustrate the validity of this approach in FIGS. 5A and 5B for g=0.6, α=0.1, which are estimated parameters of the anisotropy of scattering in tissue by Tseng et al. (S. H. Tseng, A. Grant, and A. J. Durkin, In vivo determination of skin near-infrared optical properties using diffuse optical spectroscopy, Journal of Biomedical Optics, 13(1):014016, January-February 2008.). These figures illustrate the true CDF vs. approximate CDF of HG function, along with samples generated using inversion sampling.

In some preferred implementations, the next step is to perform Hoeffding tests of independence across multiple values of μₛ. Here we have set ζ₁=Σᵢ₌₁^{M} cos (Σⱼ₌₁^{j}τⱼ sin (uⱼ)βⱼ) and ζ₂=Σ_{i=M}^{2M} cos (Σⱼ₌₁^{j}τⱼ sin (uⱼ)βⱼ).

Then, for each value of μₛ, we can sample 2000 pairs z₁, z₂ and compute the Hoeffding coefficient for the two sample sets. Two independent variables will have a Hoeffding coefficient of zero, while negatively correlated variables will have a coefficient of −½, and positively correlated variables will have a coefficient of 1. This test will give empirical support for the independence of the ζₙ. We may also analyze the sum of the ζₙ for normality. Here we have sampled 5000 values of z₁ₙₛ, where z₁ₙₛ=Σₙ₌₁^{T}ζₙ, and T is chosen so that TM corresponds to 1 nanosecond of photon travel. Thus, given M according to (30), we have T=2.25×10²/M, where the numerator corresponds to the number of millimeters light travels in water per nanosecond. Thus, Zins will be the z position of a photon after 1 nanosecond of time, given the photon travels in water.

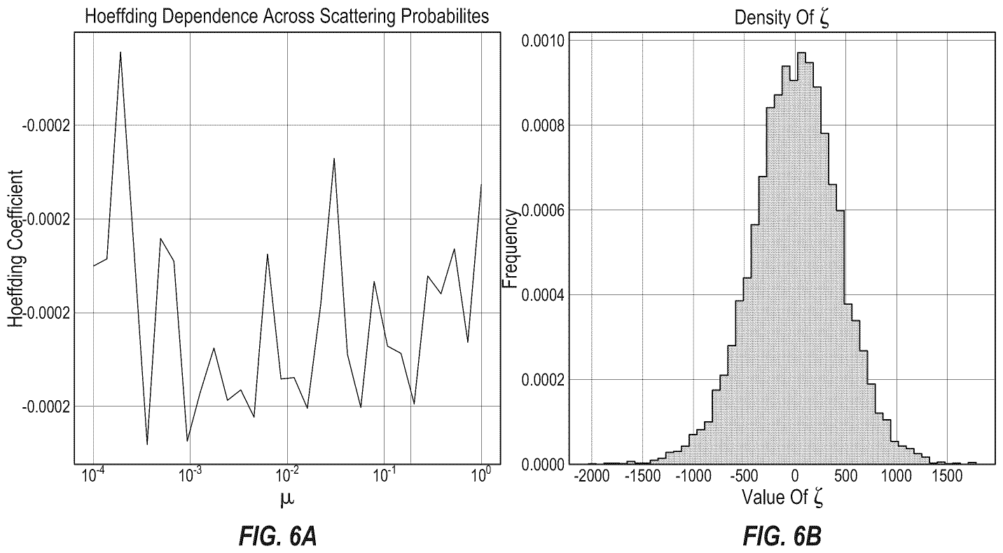

FIG. 6A is a plot of Hoeffding coefficients for independence and FIG. 6B is a histogram of the z₁ₙₛ for μₛ=1. These figures illustrate the independence of the ζₙ measured using Hoeffding coefficients, along with the density of samples from z₁ₙₛ=Σₙ₌₁^{T}ζₙ. We can see that the ζₙ are likely to be independent, and it appears that T is large enough that the z₁ₙₛ are normally distributed.

We can then test for normality of the ζₙ using the Shapiro-Wilks test. This test gives empirical support for the convergence of the zₙ to the normal distribution by CLT, which might not be a good assumption if the number ζₙ required to reach normality is very large (if M is very large, then T will be small, and so assuming the zₙ are normally distributed may not be justified.). The results below indicate that T is indeed large enough that the zₙ may be assumed to be normal, at least for values of μₛ in the range [0.1,1], as indicated by Tseng et al. The Shapiro-Wilks test will produce a p-value depending on the normality of the sample. If the p-value is large, we fail to reject the null hypothesis that the samples are normally distributed, which indicates that the samples might be normally distributed.

Below is a table of the median Shapiro-Wilks score (denoted SW) across ten tests for various μₛ in [0.1,1]. This table includes values for μₛ and the median Shapiro-Wilks test p-values. Here, we see large p-values across all of the μₛ, thus failing to indicate that indicating that the z₁ₙₛ are not drawn from a distribution that is not likely to be statistically significantly different from a normal distribution.

| μₛ | 0.1 | 0.14 | 0.2 | 0.29 | 0.42 | 0.6 | 0.86 | 1.0 |
|---|---|---|---|---|---|---|---|---|
| SW | 0.425 | 0.684 | 0.595 | 0.373 | 0.122 | 0.201 | 0.131 | 0.188 |

Given these results, we see no reason we cannot assume that the ζₙ are i.i.d, and the zₙ are normally distributed. Therefore, our lower bound M allows us to continue our analysis, and we can write:

$${z}_{n + 1} = {z}_{n} + {ζ}_{n}$$

Where the ζₙ are independent and are given by:

$${ζ}_{n} = l \cdot {\sum }_{i = 1}^{M} cos ( {\sum }_{j = 1}^{i} {τ}_{j} sin ( {u}_{j} ) {β}_{j} )$$

Assuming the independence of the ζₙ, the random walk zₙ has the Markov property, since:

$$E ( {z}_{n} \vert {z}_{n - 1} ) = E ( {z}_{n} \vert {z}_{n - 1} , {z}_{n - 2} , … , {z}_{1} )$$

As long as we pick a stopping time T for z that is a multiple of M, if we define a new process zₛ^{(T)} as:

$${z}_{s}^{( T )} = {z}_{T + s} - {z}_{T}$$

we will have the strong Markov property for zₛ^{(T)}, and so it will be independent of z_{T}. with the steps ζₛ distributed identically to the ζₙ governing the z_{T} random walk.

We also have the density of the zₙ for a fixed n is a normal distribution by CLT. Moreover, since the distribution of the ζₙ has mean zero, we will show the sample paths of the zₙ are self-similar by the strong Markov property, which allows us to use the reflection principle to derive a density for the hitting times. Much of the following work mirrors the proof of the reflection principle in Brownian Motion, Martingales and Stochastic Calculus by Jean-Francois Le Gall. (Gall, J. L. (2016), Brownian Motion, Martingales, and stochastic calculus, In Graduate texts in mathematics, https://doi.org/10.1007/978-3-319-31089-3).

Using the strong Markov property of z, we can prove the following: For every sϵN, set S_{sM}=max_{nM≤sM}z_{nM}. Then if d≥0 and bϵ(−∞, d] we have

$$P ( {S}_{s M} \geq d , {z}_{s M} \leq b ) = P ( {z}_{s M} \geq 2 d - b ) )$$

And S_{sM} has the same distribution as |Z_{sM}|. Then, by Chung-Fuchs theorem, we have that T_{d}<∞, where T_{d} is the first hitting time of zₙ for distance d. Then we have:

$$P ( {S}_{s M} \geq d , {z}_{s M} \leq b ) = P ( {T}_{d} \leq sM , {z}_{s M} ) = P ( {T}_{d} \leq s M , {z}_{s M - {T}_{d}}^{( {T}_{d} )} \leq b - {z}_{{T}_{d}} )$$

Then, since the distribution of the ζₙ is symmetric, we have the reflection principle for the random walk. Thus, we have:

$$P ( - {z}_{s M - {T}_{d}}^{( {T}_{d} )} \leq b - {z}_{{T}_{d}} ) = P ( {z}_{s M - {T}_{d}}^{( {T}_{d} )} \leq b - {z}_{{T}_{d}} )$$

Then, since the event {zₜ≥2z_{T}_{_{d}}} is contained in {T_{d}≤t}, we can write:

$$P ( {T}_{d} \leq t , - {z}_{s M - {T}_{d}}^{( {T}_{d} )} \leq b - {z}_{{T}_{d}} ) = P ( {T}_{d} \leq t , {z}_{s M} \geq 2 {z}_{{T}_{d}} - b ) = P ( {z}_{s M} \geq 2 {z}_{{T}_{d}} - b )$$

Note that P(Bₜ≥2d−b) is the equivalent statement in the case of a Brownian Bₜ. Note, in the Brownian case, since uncountably many random Gaussian variables generate the process, B_{T}_{_{d}}=d is justified by the continuity of sample paths. Here, since random vector defining the process zₙ given by (ζ₁, ζ₂, . . . ζₙ) is countable, we cannot say that z_{T}_{_{d}}=d, but that z_{T}_{_{d}}≥d. However, because the variation of the ζₙ is bounded, we can say that:

$$P ( {z}_{{T}_{d}} - d > ε ) < δ ,$$

for some small ϵ, δ. Then we can say:

$$P ( {z}_{sM} \geq 2 {z}_{{T}_{d}} - b ) \approx P ( {z}_{sM} \geq 2 d - b )$$

Then we have:

$$P ( {S}_{sM} \geq d ) = P ( {S}_{sM} \geq d , {z}_{sM} \geq d ) + P ( {S}_{sM} \geq d , {z}_{sM} \leq d ) = 2 P ( {z}_{sM} \geq d ) = P ( \vert {z}_{sM} \vert \geq d ) ,$$

where the third equality follows from the fourth equation above, and {z_{sM}≥d}⊂{S_{sM}≥d}. We can now derive the density of the T_{d}. We first characterize Σᵢ₌₁ⁿζᵢ to define the variance of the zₙ. We have that for each ζᵢ, each step is taken according to the update equations above, where we take small steps l corresponding to a small distance along the path of z. These small steps in distance correspond to a small step in time, according to the velocity of light in the medium. Then, given l<<1, we have that 1/l is the number of steps taken at time t=1 (note in our calculation of the ζₙ, our choice of l impacts μₛ.). Then we have

$$l = \frac{1}{l M}$$

is the number of ζₙ steps we take by time t=1. Then we have that Σₗ₌₁^{l}ζᵢ will be approximately normally distributed by CLT and will have some variance σ².

Then, by the property of normal distributions that samples xⱼ from N(0,σ²) satisfy Σⱼ₌₁ⁿxⱼ˜N(0, nσ²), and \sqrt{n}xⱼ˜N(0, nσ²), we have that zₙ has the same distribution as

$$\sqrt{\frac{n}{l}} {z}_{l} .$$

Thus, using the equation above, we have:

$$P ( {T}_{a} \leq sM ) = P ( {S}_{sM} \geq d ) = P ( \vert {z}_{sM} \vert \geq d ) = P ( {z}_{sM}^{2} \geq {a}^{2} ) = P ( \frac{sM}{l} {z}_{l}^{2} \geq {d}^{2} ) = P ( \frac{{d}^{2}}{{z}_{l}^{2}} \leq \frac{sM}{l} )$$

Then, since z, is distributed according to N(0, σ²), we can calculate

$$P ( \frac{{d}^{2}}{{z}_{l}^{2}} \leq \frac{sM}{l} ) .$$

We consider

$$g ( z ) = \frac{{d}^{2}}{{z}_{l}^{2}} ,$$

and so

$${g}^{- 1} ( x ) \sqrt{\frac{{d}^{2}}{x}} .$$

Since g is decreasing on its range, we have g⁻¹ is likewise decreasing. Then we have

$$\{ g ( z ) \leq \frac{sM}{l} \} = \{ z \geq {g}^{- 1} ( \frac{sM}{l} ) \}$$

and so we have

$$P ( g ( z ) \leq \frac{sM}{l} ) = P ( z \geq {g}^{- 1} ( \frac{sM}{l} ) ) .$$

Then, we have

$$P ( z \geq {g}^{- 1} ( \frac{sM}{l} ) ) = 1 - P ( z \leq {g}^{- 1} ( \frac{sM}{l} ) ) ,$$

and thus we have via u-substitution with

$$u = {g}^{- 1} ( z ) = \sqrt{\frac{{a}^{2}}{z}} ,$$

gives:

$$1 - {\int }_{- ∞}^{\sqrt{\frac{{ld}^{2}}{sM}}} \frac{1}{\sqrt{2 π {σ}^{2}}} {e}^{\frac{- {x}^{2}}{2 {σ}^{2}}} dx = - {\int }_{\frac{sM}{l}}^{0} \frac{1}{2} \frac{d}{\sqrt{2 π {σ}^{2}}} {u}^{- 3 / 2} {e}^{- \frac{{d}^{2}}{2 u {σ}^{2}}} du$$

But since P(|z|>d)=2P(z>d), we can normalize the above expression by multiplying by 2, which accounts for the concentration of probability mass due to

$$\frac{{d}^{2}}{{z}^{2}}$$

mapping all values of z into [0, ∞).

Thus, we have that the density of hitting times Tₐ is given by:

$${f}_{{T}_{d}} ( t ) = ℒ ( t \vert d , σ ) = \frac{d}{\sqrt{2 π {σ}^{2}}} {t}^{- 3 / 2} {e}^{{d}^{2} / ( 2 t {σ}^{2} )}$$

Where we have replaced

$$\frac{s M}{ι}$$

by t for simplicity.

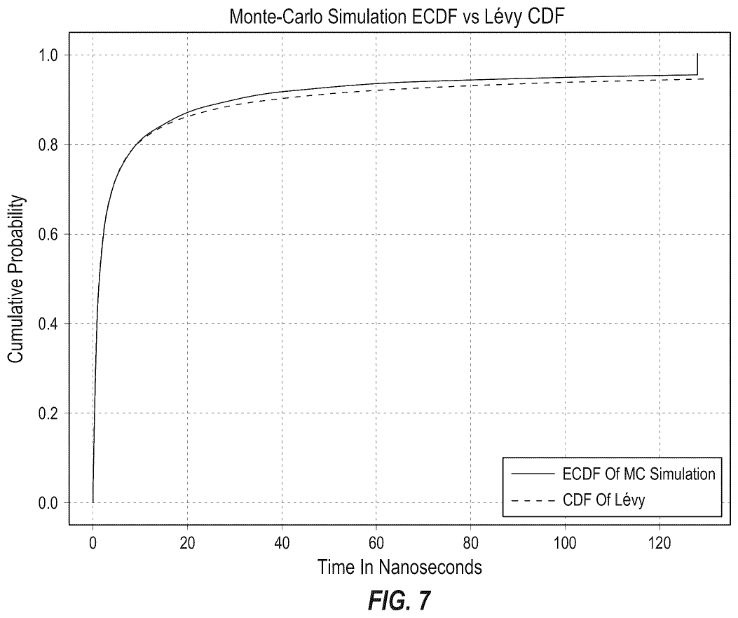

FIG. 7 provides empirical CDF of samples generated from Monte-Carlo simulation of the photon update equations compared to the modified Levy distribution, using σ learned from the MC data with a PyMC Bayesian sampler.

To test the analysis above, we generate samples from HG(ψ|g,α), Bernoulli(μₛ), and Uniform (0,2π), and simulate 20,000 photons for 5×10⁸ steps, simulated using a photon scattering algorithm. The algorithm was implemented in Jax, using the vmap parallelization functionality and just-in-time compilation features. 20,000 photon packets were simulated for 5×10⁸ steps in 20 minutes on an RTX-3090.

Here, each photon consists of an x, y, z coordinate, a current heading, a photon strength value, and a hitting time value. We start each photon with a strength of 100, and every time a photon is absorbed, we subtract 1 from the strength. Photon positions are updated according to the update equations referenced above.

We then calculate the cumulative probability distribution of the hitting times at z=0 for the Monte-Carlo particles and compare it to the CDF of the modified Levy distribution with parameters d=1, and σ=1.29 learned by Bayesian estimation from the Monte-Carlo samples. The two are plotted in FIG. 7.

We note here the uptick in samples at the very end. This is due to the simulation time being capped at 5×10⁸ steps. If we were to simulate for longer, it would be reasonable to expect the tails to match up correctly, given a large enough number of particles. Here, we note the maximum error between this final value of the MC samples and the Levy distribution is 0.009 or about 17%. This error is should be acceptable for most purposes, as larger times mean more attenuation, so error from tail photons is negligible.

These results support the assumption that the modified Levy distribution describes the hitting times T_{d}, which correspond precisely to the Aⱼ in the equation above. Thus, we have a simple summation to represent the incident light at any particular wavelength, and since we can describe εⱼ and Aⱼ with probability density, we can further approximate the summation to give a closed-form model for incident light.

It may be desirable to further provide an integrated recursive path approximation. If we wish to approximate the sum [IRPA_SUM], using two terms, and account for the time-varying nature of the signal, due to the pulse of the blood. We wish to split up the sum based on path length, and after more analysis, we can show:

$${I}_{{D}_{t}} = \frac{{I}_{0}}{N} {\sum }_{j}^{N} {e}^{- {lA}_{j} {\overline{ε}}_{{j}_{t}}} \approx {I}_{0} {L}_{1} {e}^{- {cA}_{1} {ε}_{T}} + {I}_{0} {L}_{2} {e}^{- {cA}_{2} ( ( 1 - p ( t ) ) {ε}_{B} + p ( t ) {ε}_{T} )}$$

Note the subscript t on εⱼ_{ₜ}, which denotes the time dependence of the εⱼ on t, since we now consider the intensity across time. Aⱼ has no dependence on time since Aⱼ determines how long a photon path spends in the body, not what it encounters. εⱼ accounts for this change in path absorption across time.

We have that L₁, L₂ sum to 1 and are the integrals of the modified Levy distribution from 0 to some time τₛ corresponding to paths that have a near zero probability of entering the artery, and the integral of the modified Levy from τₛ to ∞, respectively. Thus, we have:

$${L}_{1} = {\int }_{0}^{{τ}_{s}} ℒ ( t \vert d , σ ) d τ$$

$${L}_{2} = {\int }_{{τ}_{s}}^{∞} ℒ ( t \vert d , σ ) d τ$$

Then A₁, A₂ are the hitting times of the average detected path over the two sections of the Levy distributions, respectively. Thus, we have:

$${e}^{- c {A}_{1} {ε}_{B {T}_{1}}} = \frac{1}{{L}_{1}} {\int }_{0}^{{t}_{s}} {e}^{- ct {ε}_{B {T}_{1}}} ℒ ( t \vert d , σ ) dt$$

$${e}^{- c {A}_{2} {ε}_{B {T}_{2}}} = \frac{1}{{L}_{2}} {\int }_{{t}_{s}}^{∞} {e}^{- ct {ε}_{B {T}_{2}}} ℒ ( t \vert d , σ ) dt$$

where ε_{BT}_{₁}, ε_{BT}_{₂} are expected bulk absorption in each path bin, respectively. Note we scale by 1/L₁, 1/L₂ respectively so each probability density measure over the subset of path lengths still integrates to 1.

Finally, p(t) is some function that determines what percentage of time the A₂ path spends in tissue vs blood, dependent on the pulse of the blood, which is dependent on time.

Note tₛ and p(t) are codependent since tₛ determines A₁, A₂, L₁, L₂. To expand the analysis on p(t), we can decompose it as p(t)=a·f(t)+b, where f(t) is strictly a function of the pulse, which models the volumetric change of blood in the area of the body we monitor. We have that b is an offset modeling the percentage of time paths spend in the tissue at both systolic and diastolic blood volumes, and a is a scalar that parameterizes the change in percentage of time paths spend in the blood between systolic and diastolic blood volumes.

Here, we note that b is a proxy for the distance of the artery from the skin. Then, given some parameter [symbol] (which might have to be learned) we have for the distance parameter d in the Levy distribution: d= [symbol] ·b. We can also define α as an offset parameter so that paths start some distance α into the skin. This is done so the Levy distribution does not collapse to a Dirac distribution. For most purposes, it is sufficient to set α to 1 (assuming a length unit of millimeters).

Then, given d, we may determine an upper bound on the probability that a path of length s enters the artery. We define this function L:

$$ℒ ( s \vert d , α , σ ) = {sup}_{\begin{matrix}{t}_{1} , {t}_{2} \vert \\ {t}_{1} + {t}_{2} = s\end{matrix}} \{ {\int }_{0}^{{t}_{1}} ℒ ( t \vert d , σ ) {\int }_{0}^{{t}_{2}} ℒ ( t \vert d + α , σ ) \}$$

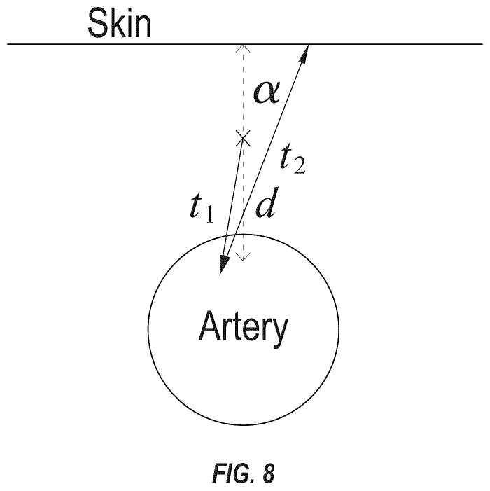

Note here this expression is similar to a previous equation, but we wish to consider not zₜ only at t=sM, but inf_{t≤sM}{zₜ}, as we could have zₜ enter the artery at some time t<sM, but then rise back above the level of the artery. By self-similarity we can consider the suprema of two shorter paths, which leads to our definition of [symbol] . To justify this definition, we can consider the diagram of FIG. 8.

In FIG. 8, the t₁ corresponds to the t₁ in the upper bound of integration on the leftmost integral in the definition of [symbol] , and t₂ likewise on the rightmost integral in the definition of [symbol] .

Then [symbol] (s) computes the maximal probability that a path both enters the artery and returns to the skin, given that the total path length must be less than s. We consider two sub-paths, where the first path represents the distance from the original path's starting point, and the second is the distance from the artery to the skin. This reduces to evaluating the density of hitting times of paths of distance d corresponding to the t₁ path and the density of hitting times of paths of distance d+α, corresponding to the t₂ path. Then, the product of these two integrals gives the probability that a path with length less than or equal to t₁ hits distance d and that a path of length less than or equal to t₂ hits distance d+α.

Then, because the paths exhibit self-similarity and the strong Markov property, we have independence between time steps, so we can factor the probability that a path with length less than or equal to s both hits the artery and the skin as the product of the probability that two paths hit the artery, and the skin, which leads to the expression involving the product integrals over the Levy density. This gives [symbol] (s) as the upper bound on this probability for a given path length s.

Then once d is determined, we have that given σ, we may compute tₛ as:

$${t}_{s} = {sup}_{s} \{ s \vert ℒ ( s \vert d , α , σ ) < δ \}$$

Where δ is some small value, which indicates the probability that a path of length tₛ has at most probability δ of entering the artery and returning to the skin.

Then tₛ is determined given b, and once tₛ is known, then A₁, A₂, L₁, L₂ are determined, which necessarily determines a in the expression p(t)=a·f(t)+b, given f(t).

Preferred embodiments and implementations may utilize machine learning models that exploit this new understanding to extract parameters of interest, such as ε_{B}. This is because ε_{B} is a weighted average of ε_{g}, εₕ . . . , which are the extinction coefficients of red blood cells and other blood parameters, and the weights on those coefficients correspond to the absolute concentration of those parameters of interest. In some cases, the common term I₀L₁e^{-cA}^{₁}^{ε}^{_{T}} may be isolated, which might be done using the loss function:

$$arg {min}_{x , y} var ( \frac{\frac{d}{dt} log ( {I}_{{D}_{1 t}} - x ) / x}{\frac{d}{dt} log ( {I}_{{D}_{2 t}} - y ) / y} ) + var ( \frac{\frac{d}{dt} log ( {I}_{{D}_{2 t}} - y ) / y}{\frac{d}{dt} log ( {I}_{{D}_{1 t}} - x ) / x} )$$

where I_{D}_{₁ₜ}, I_{D}_{₂ₜ}, correspond to the detected luminance at time t for wavelengths 1 and 2, respectively. Here var is the sample variance function, which should be minimized when x and y correspond to I₀_{₁}L₁e^{-cA}^{₁}^{ε}^{_{T1}}, I₀_{₂}L₁e^{-cA}^{₁}^{ε}^{_{T2}}, respectively. This is because the fraction:

$$\frac{\frac{d}{d t} log ( {I}_{{D}_{1 t}} - {I}_{{0}_{1}} {L}_{1} {e}^{- c {A}_{1} {ε}_{{T}_{1}}} ) / {I}_{{0}_{1}} {L}_{1} {e}^{- c {A}_{1} {ε}_{{T}_{1}}}}{\frac{d}{d t} log ( {I}_{{D}_{2 t}} - {I}_{{0}_{2}} {L}_{1} {e}^{- c {A}_{1} {ε}_{{T}_{2}}} ) / {I}_{{0}_{2}} {L}_{1} {e}^{- c {A}_{1} {ε}_{{T}_{2}}}}$$

evaluates to

$$\frac{{ε}_{{B}_{1}} - {ε}_{{T}_{1}}}{{ε}_{{B}_{2}} - {ε}_{{T}_{2}}} ,$$

which should be constant over a short period of time, and so should have zero variance over a short time (one or two heartbeats). One might replace the sample variance by the information-theoretic differential entropy function, as a constant value will have zero entropy (Dirac distribution).

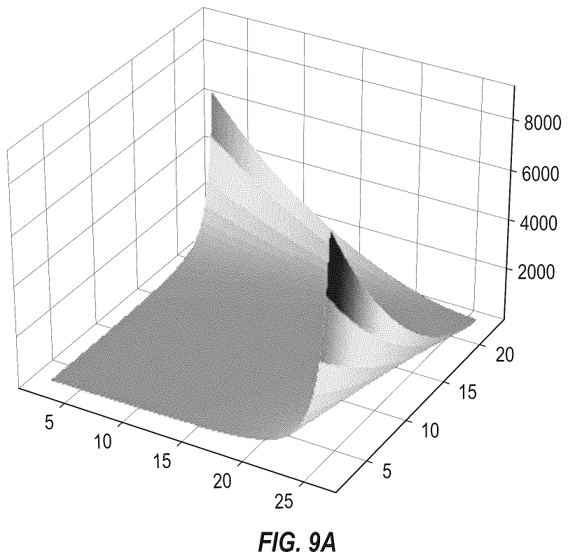

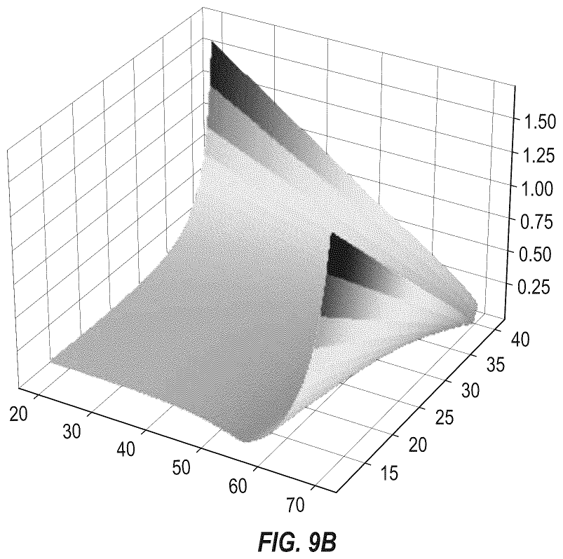

Experimental support for the integrated model disclosed herein has been established. Initial results on real data using the loss function defined above are described below and are shown in FIGS. 9A and 9B. We gathered real world spectrometer data in order to test the effectiveness of the integrated model developed above.

The experimental data I_{D}_{_{1,t}}, I_{D}_{_{1,t}} are taken from an Hamamatsu InGaAs (Indium Galium Arsenide) infrared spectrometer developed by Tula Health, Inc. in conjunction with the Brigham Young University Physics Department. To produce the data used below, the spectrometer was placed against the radial artery in the wrist of a test subject, and data were recorded to facilitate further analysis.

Given this data, we choose I_{D}_{₁} at wavelength 1300 nm, and I_{D}_{₂} at wavelength 1100 nm. In FIG. 9A, we see the loss plotted for various values of x, y corresponding to various DC offset values.

In FIG. 9B, we see a similar loss surface generated for synthetic data generated using the equations and techniques described herein. Here, the absolute magnitudes of the loss surface are irrelevant, as the synthetic data have less noise than the experimental data, which increases the variance for all loss values, so we only consider the relative magnitudes of the real vs. synthetic data.

In FIGS. 9A and 9B, it can be seen that the loss surfaces of the experimental and synthetic data are qualitatively similar, which is an indication that the integrated model captures much of the structure of experimental data. This suggests that the integrated model is representative of much of the structure seen in time dependent photon scattering measurements.

### Lemma 2:

The sum of two uniform random variables supported on the unit circle will be uniformly distributed.

### Proof:

Because circular convolution is equivalent to identifying the endpoints of the convolution interval, we may simply compute the circular convolution of two uniform random variables.

Thus, let X and Y be independent random variables, each uniformly distributed on the interval [0,1). The circular convolution of these two distributions is defined as the distribution of the random variable Z=X+Y mod 1.

The probability density function (pdf) of a uniform distribution on [0,1) is given by:

$${f}_{X} ( x ) = {f}_{Y} ( y ) = \{ \begin{matrix}1 & if 0 \leq x < 1 , \\ 0 & otherwise\end{matrix} .$$

The pdf of the sum Z=X+Y (before taking the modulo operation) is given by the convolution of f_{X} and f_{Y}:

$${f}_{Z} ( z ) = {\int }_{- ∞}^{∞} {f}_{X} ( x ) {f}_{Y} ( z - x ) d x .$$

Since f_{X}(x)=f_{Y}(x)=1 for xϵ[0,1) and 0 otherwise, the convolution integral simplifies to:

$${f}_{Z} ( z ) = {\int }_{max ( 0 , z - 1 )}^{min ( z , 1 )} 1 dx = min ( z , 1 ) - max ( 0 , z - 1 ) .$$

Now, we need to consider the modulo operation to obtain the pdf of Z mod 1, which we will denote as f_{Z %1}(z). The pdf f_{Z %1}(z) is periodic with period 1, so we only need to consider z in the interval [0,1):

For 0≤z<1, f_{Z %1}(z)=f_{Z}(z)+f_{Z}(z+1). In this interval, f_{Z}(z)=z and f_{Z}(z+1)=0, so f_{Z %1}(z)=z+0=z.

For 1≤z<2, f_{Z %1}(z−1)=f_{Z}(z−1)+f_{Z}(z). In this interval, f_{Z}(z−1)=0 and f_{Z}(z)=1−(z−1)=2−z, so f_{Z %1}(z−1)=0+(2−z)=2−z.

Combining these two cases, we find that the pdf of Z mod 1 is constant and equal to 1 for zϵ[0,1), which is the pdf of a uniform distribution on [0,1). Therefore, the circular convolution of two uniform distributions is also uniform.

Input: Boundary conditions, absorption and scattering probabilities, path length l, time step dt, scattering parameters g, α Output: Updated particle state, final particle.

Sample action from categorical distribution Determine new direction using particle position=particle position=subtract 1 from photon strength particle position=

Generate sampling u₁, u₂ from Uniform(0,1), Uniform(0,2π) Find β by computing HG⁻¹(u₁|g, α) Update new heading by computing ϕ_{new}, θ_{new} from βcos (u₂), β sin (ϕ) new direction
compute direction ϕ, θ as current particle heading+new heading compute new position as x_{new}=x_{old}+lsin (θ) cos (ϕ) y_{new}=y_{old}+lsin (θ) sin (ϕ), z_{new}=z_{old}+lcos (θ) x_{new}, y_{new}, z_{new}

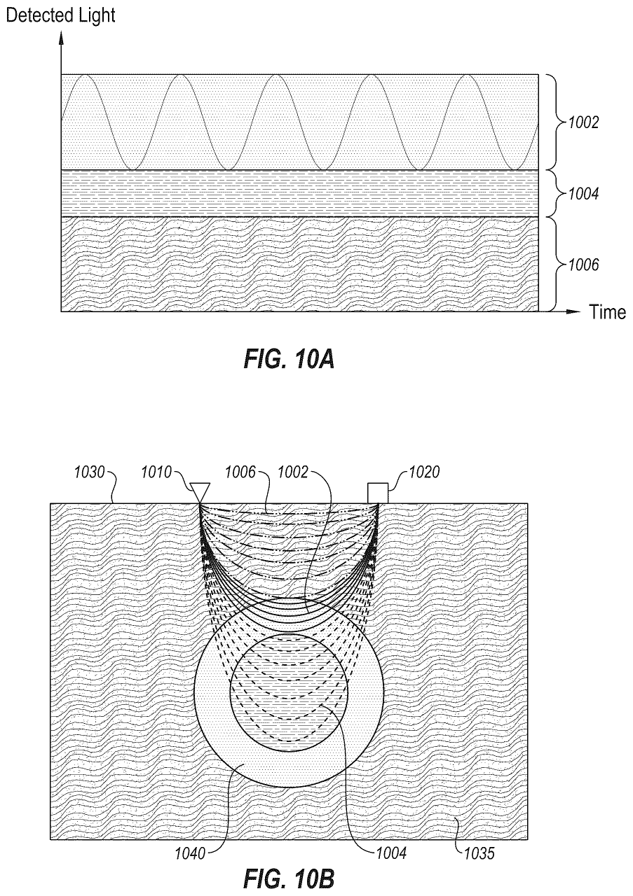

FIGS. 10A and 10B illustrate schematically a system for using electromagnetic radiation from an electromagnetic emitter 1010 to process signals and track various physiological conditions. As shown in FIG. 10B, radiation is emitted from emitter 1010 and is directed through the skin 1030 of a user, through various tissue layers 1035, and into a blood vessel 1040. The radiation is reflected at various points and received by a sensor 1020, which may comprise, for example, a spectrometer. Thus, the signal data referenced throughout this disclosure may be received by a physiological sensor, such as a non-invasive blood monitor. Examples of such monitors can be found in U.S. Pat. No. 11,903,686 titled “Systems, Apparatuses, and Methods for Optimizing a Physiological Measurement Taken From a Subject,” the entire contents of which are hereby incorporated herein by reference in its entirety.

As shown in FIG. 10B, the sensor 1020 may be configured to measure the magnitude/intensity of the received radiation over time. The resulting signal may therefore consist of a pulsatile component 1002, which corresponds to radiation 1002 being reflected from blood vessel 1040, as shown in FIG. 10B. Because the blood vessel 1040 contracts and expands at a rate corresponding with the heartbeat of the user, this portion of the signal may contain a periodic and/or pulsatile signal, as indicated by the sine wave within section 1002 of the signal.

The signal may also include a non-pulsatile component 1004 that results from radiation reflected from within the blood vessel 1040. In systems configured to predict, estimate, and/or detect blood analytes, non-pulsatile blood component 1004, otherwise referred to herein as the “baseline” component, is likely to contain useful information that may be used to provide a better prediction or estimation of the blood analyte under consideration.

The signal may further comprise a DC offset or tissue component 1006. DC offset/tissue component 1006 is the result of radiation being reflected from non-blood tissue 1035 of the user. Again, if the desired result is to assess blood analyte conditions, this portion of the signal is not likely to contain useful information. Moreover, having this portion of the signal present may detract from the useability of components 1002 and 1004. This is because prior techniques for processing data with all three components 1002/1004/1006 fail to differentiate between components 1004 and 1006. For example, it has been typical to simply assume that components 1004 and 1006 were both resulting from tissue reflections, and therefore did not contain useful information. However, the techniques provided herein allow for not only differentiation of components 1004 and 1006, but also for extraction of one or both of these components. For example, it may be desirable to extract DC offset/tissue component 1006 entirely so that it does not detract from the usefulness of the information in the other two components 1002 and 1004.

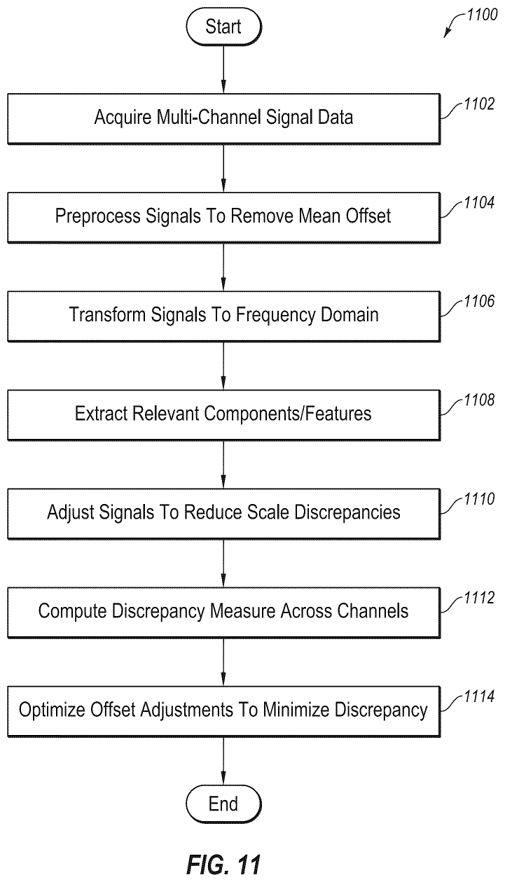

FIG. 11 is a flowchart illustrating an example of a method 1100 for processing signal data to estimate, calculate, and/or predict a condition, such as a blood analyte condition, according to some implementations. At step 1102, signal data may be received. In some cases, the signal data may be multi-channel signal data. For example, each channel of such a multi-channel signal may correspond with a distinct wavelength, wavelength range, and/or sensor. This may provide for enhanced detection capabilities by taking advantage of different absorption characteristics of glucose or other analytes at various wavelengths, typically within the near-infrared (NIR) spectrum. As previously mentioned, the signal data may be received from a blood monitoring device and/or system, which may comprise a wearable device, such as a wrist monitor, that may be configured with the aforementioned emitter(s) and sensor(s).

Although use of a single channel of signal data may be feasible for some uses, a multi-channel signal may allow for capturing the signal Si(t) at different settings.

According to the preferred embodiments, these signals may have the functional form: Si(t)=Ci+Di·exp(−vi·p(t)), where Ci is the offset value for each channel I, Di is an unknown scaling factor, vi is a vector of weights dependent on the channel i. and p(t) is a pulse function representing the physiological signal.

By collecting multi-channel data, multiple observations of the same underlying physiological process may be accessed and/or may be modulated differently according to the weights vi. This setup allows leveraging the relationships between channels to extract the common offset C and improve signal processing.

At step 1104, the incoming signals may be processed to remove the mean offset. In some implementations and embodiments, the offset C may be removed from each signal by subtracting a proposed offset value c. Since C is unknown, in some cases, one could start with an initial guess c and adjust it through optimization. After subtracting c, the natural logarithm of the result may be taken. According to the preferred implementations of the model, this yields: log (Si(t)−c)=log (D)−vi·p(t).

Since D is unknown and acts as an additive constant in the logarithmic domain, in some cases it can be ignored, instead focusing on frequency components greater than zero.

Subtracting c aims to estimate and remove the offset C. Taking the logarithm linearizes the exponential function in the model, transforming the multiplicative relationship into an additive one. Ignoring log (D) (the DC component) in subsequent frequency-based analysis allows us to compare the dynamic parts of the signals across channels.

The signals may then be transformed to the frequency domain at step 1106. In some implementations, the logarithmically transformed signals may be converted into the frequency domain using the Fast Fourier Transform (FFT). This transformation enables the analysis of frequency components associated with the pulsatile function p(t).

Although not required for all contemplated embodiments and implementations, transforming to the frequency domain isolates the frequencies related to p(t). Removing the DC component (log (D)) ensures focus on the variations caused by −vi*p(t), which differ across channels due to different vi values.

One or more features, parameters, and/or components may then be extracted from the data at step 1108. In some cases, this may be done via machine learning. In some implementations, step 1108 may comprise excluding the zero frequency (DC) component and/or the highest frequency component, which may allow for concentrating on the frequencies that carry the pulsatile information. This step may also isolate the relevant features influenced by p(t) and vi.

By removing the zero-frequency component, one can effectively eliminate log (D), a constant offset. Focusing on non-zero frequencies allows for analyzing the dynamic behavior of the signals due to p(t).

The signal(s) may then be adjusted, aligned, and/or normalized at step 1110. In some cases, step 1110 may comprise reducing scale discrepancies in the signal(s). In some cases, the signals may be normalized and aligned to account for sign differences in vi. Since vi may have different signs across channels, aligning ensures that the signals are comparable. Normalization may remove differences in scale, allowing direct comparison of signal shapes.

Alignment may be useful for correcting discrepancies due to different signs of vi. Normalizing the signals removes scaling differences, ensuring that any remaining variance is due to incorrect offset c rather than amplitude differences.

Step 1112 may comprise computing discrepancy measure and/or variance across the signal channels. After alignment and normalization, the variance across channels may be computed, preferably at each frequency. If c=C, the transformed signals should be identical across channels, resulting in zero variance. Nonzero variance indicates that the offset c is incorrect.

Because the variance serves as a discrepancy measure or loss function, minimizing this variance helps find the value of c that best estimates the true offset C, thereby leveraging the model's relationships between channels.

The offset adjustments may then be improved and/or optimized to reduce and/or minimize discrepancy and/or variance at step 1114. In some implementations and related embodiments, the computed variance (loss function) may be used in an optimization routine to adjust the proposed offset c. By iteratively updating c to minimize the loss, convergence towards the true offset C may be achieved.

Optimization techniques (e.g., gradient descent) may use the loss to update c. Since the loss is differentiable with respect to c, gradients can be computed and the c that minimizes the variance, or at least reduces the variance, can be identified, thereby allowing for more accurately estimating C.

Method 1100 may conclude when the optimization converges and the variance reaches a minimum value, or a predetermined minimum threshold value.

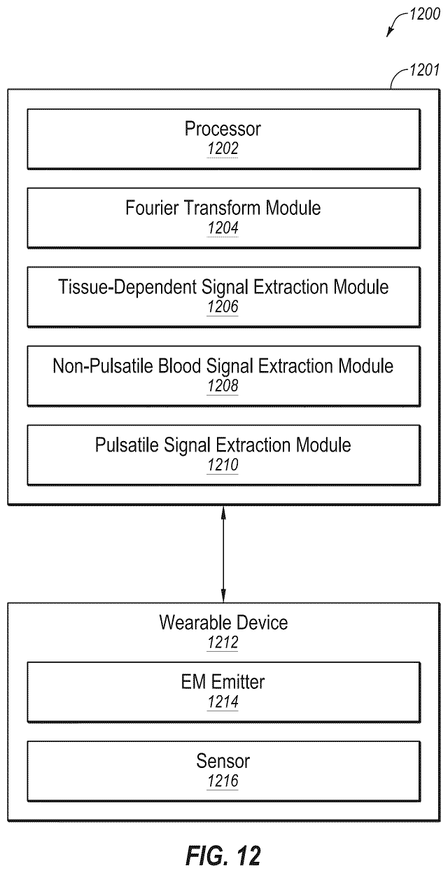

FIG. 12 is a schematic diagram of a system 1200 for processing data, such as data from a wearable device 1212 used for monitoring health conditions, such as blood analyte conditions, according to some implementations. As shown in this figure, wearable device 1212, which may comprise a wrist monitor in some embodiments, comprises one or more electromagnetic emitters 1214 and one or more sensors 1216 configured to receive reflected radiation from the emitter(s) 1214. In some embodiments, the wearable device 1212 may be positioned on the wrist so that a physiological sensor 1216 is positioned adjacent to a particular, targeted physiological feature, such as an ulnar artery or radial artery, for example.

In some embodiments, an electromagnetic emitter 1214 may be positioned at a suitable location relative to the sensor1 216 so that electromagnetic radiation of a desired wavelength may be emitted from emitter 1214, reflected from desired regions within the body, such as along an adjacent to a particular artery in the case of blood monitoring, and then received by sensor 1216 for processing according to, for example, the method of FIG. 11 or any of the other methods disclosed herein.

As those of ordinary skill in the art will appreciate, sensor 1216 may be configured to convert reflected/incoming radiation into electronic signals, which may be transmitted to and received by processing system 1201. For example, in some embodiments, sensor 1216 may be configured to measure the intensity of the received radiation over time and transmit this signal to system 1201. System 1201 may be incorporated onto the wearable device 1212 or may be incorporated into another device, such as a mobile application running on a smartphone, a computer, a remote server, or the like.

System 1201 may comprise one or more processors 1202. Processor(s) 1202 may be configured to receive and/or process data from wearable device 1212 and/or sensor 1216. In addition, processor(s) 1202 may be configured to perform various functions, some of which may be part of a particular software module. As used herein, a software module or component may include any type of computer instruction or computer executable code located within a memory device and/or m-readable storage medium. A software module may, for instance, comprise one or more physical or logical blocks of computer instructions, which may be organized as a routine, program, object, component, data structure, etc., that perform one or more tasks or implements particular abstract data types.

System 1201 may comprise a Fourier Transform module 1204. Fourier Transform module 1204 may be configured to take raw and/or preprocessed data, which preferably includes a periodic feature, and project the signal data onto a set of basis functions. In some embodiments, Fourier Transform module 1204 may be configured to extract one or more features of a periodic aspect of the signal, such as features associated with a shape of a pulsatile signal contained within the data. In some embodiments, certain frequency amplitudes computed by the Fourier Transform Module 1204 might be indicative of certain analyte concentrations, but also may be indicative of certain cardiovascular conditions. It may therefore be possible to diagnose, for example, atrial fibrillation or similar conditions by analyzing the shape of the waveform with Fourier analysis.

System 1201 may further comprise a Tissue-Dependent Signal Extraction module 1206. In some embodiments, Tissue-Dependent Signal Extraction module 1206 may be configured to utilize one or more of the steps described above in connection with method 1100 to extract a DC offset portion of a signal, which in some cases may be the portion of the signal corresponding with reflections from non-blood tissue, or may otherwise comprise portions of the signal that may not be needed and/or may be detrimental to providing accurate assessments of blood analyte content. In some embodiments, Tissue-Dependent Signal Extraction module 1206 may therefore be configured to use one or more of the DC extraction methods described herein.

System 1201 may further comprise a Non-Pulsatile Blood Signal Extraction module 1208. In some embodiments, module 1208 may be configured to extract a portion of the signal corresponding to reflections from the blood itself rather than the blood vessel wall. In some cases, module 1208 may comprise simply compiling the data remaining following extraction of the DC offset portion of the signal, along with the pulsatile signal (described below).

Some embodiments may further comprise a Pulsatile Signal Extraction module 1210. In such embodiments, module 1210 may be configured to extract the portion of the signal that comprises a periodic and/or pulsatile signal received by sensor 1216. In embodiments in which system 1200 is used to provide blood analyte condition data, such as glucose monitoring data, module 1210 may therefore be configured to extract the portion of the signal associated with a heartbeat signal or other physiological, periodic signal. As those of ordinary skill in the art will appreciate, in some embodiments, module 1210 may utilize Fourier transforms and/or analyses, such as the Fast Fourier Transform (FFT). In some embodiments and related implementations, once the DC offset has been extracted, as described throughout this disclosure, the remaining signal, which comprises a pulsatile signal and a non-pulsatile portion, which may comprise a non-pulsatile blood portion in some cases, may be processed to separate them by simply extracting the offset from the remaining signal and/or extracting the pulsatile portion, either of which may be accomplished in a relatively straightforward manner.

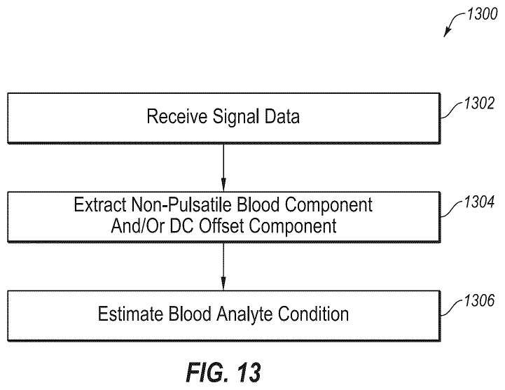

Another method 1300 according to some implementations is illustrated in the flowchart of FIG. 13. In this method, signal data is received at step 1302. In some implementations, the signal data may comprise multi-channel signal data. In some implementations, the signal data may be received from a physiological sensor and/or monitor, such as a blood glucose monitor or a pulse oximeter, for example.

The signal data may then be processed at step 1304 to extract one or more components. For example, in some implementations in which blood analytes, such as oxygen or glucose, are being monitored, the signal may comprise useful data and non-useful data. If the device that is providing the data signal is configured to use electromagnetic radiation directed towards a blood vessel, for example, it may be useful to extract a component, or at least as much of a component as possible, that corresponds to reflected radiation from tissue rather than blood and/or blood vessels. This component may otherwise be referred to herein as a DC offset component. Techniques for extracting this component are taught throughout this disclosure.

In addition, for some purposes, it may be desirable to extract a non-pulsatile blood component from the signal. As previously mentioned, this component may correspond with reflected radiation from the blood itself within a blood vessel. Because techniques for extracting the pulsatile signal and the DC offset/tissue component are taught herein, the remaining portion of the signal may be considered the non-pulsatile blood component and may therefore be extracted by extracting the other two components. It is thought that this component may be used alone for certain purposes.

A condition, such as a blood analyte condition, may then be calculated, predicted, estimated, and/or assessed at step 1306. In some implementations, this step may be performed by use of machine learning algorithms. Additionally, or alternatively, this step may be performed by calibrating the processed and/or extracted data, such as by calibrating results with more accurate and/or invasive techniques for assessing blood analyte conditions. In some cases, calibration may be achieved by finding blood analytes using known lab procedures, such as metabolic panels and the like, which may then be compared with the results of the modeling and/or processed data.

In certain embodiments, a particular software module may comprise disparate instructions stored in various locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices.

In addition, data being tied or rendered together in a database record may be resident in the same memory device, or across several memory devices, and may be linked together in fields of a record in a database across a network. Furthermore, embodiments and implementations of the inventions disclosed herein may include various steps, which may be embodied in machine-executable instructions to be executed by a general-purpose or special-purpose computer (or another electronic device). Alternatively, the steps may be performed by hardware components that include specific logic for performing the steps, or by a combination of hardware, software, and/or firmware.

Embodiments and/or implementations may also be provided as a computer program product including a machine-readable storage medium having stored instructions thereon that may be used to program a computer (or other electronic device) to perform processes described herein. The machine-readable storage medium may include, but is not limited to: hard drives, floppy diskettes, optical disks, CD-ROMs, DVD-ROMs, ROMs, RAMs, EPROMS, EEPROMs, magnetic or optical cards, solid-state memory devices, or other types of medium/machine-readable medium suitable for storing electronic instructions. Memory and/or datastores may also be provided, which may comprise, in some cases, non-transitory machine-readable storage media containing executable program instructions configured for execution by a processor, controller/control unit, or the like.

It will be understood by those having ordinary skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. The scope of the present invention should, therefore, be determined only by the following claims.

1. A method for processing signals from a non-invasive blood analyte tracking system, the method comprising the steps of:
receiving signal data from a non-invasive blood monitor, wherein the signal data comprises:
a frequency component corresponding to a heart rate pulse of a user of the non-invasive blood monitor;
a non-pulsatile blood component corresponding to light reflected from within a blood vessel; and
a tissue-dependent DC offset component;
processing the signal data to extract the tissue-dependent DC offset component; and
evaluating the processed signal data to provide an estimate of a blood analyte condition using only the frequency component and the non-pulsative blood component from the signal data.

2. The method of claim 1, wherein the blood analyte condition comprises a concentration of the blood analyte.

3. The method of claim 1, wherein the blood analyte condition comprises a trend over time associated with the blood analyte.

4. The method of claim 1, wherein the blood analyte comprises glucose.

5. The method of claim 1, wherein the step of processing the signal data comprises use of a probability density model.

6. The method of claim 5, wherein the probability density model comprises a Levy Distribution.

7. The method of claim 1, wherein the blood analyte comprises oxygen.

8. A method for extracting a DC offset component from signal data using a non-invasive blood monitor, the method comprising the steps of:
receiving signal data from a non-invasive blood monitor;
processing the signal data to remove a mean offset component from the signal data;
transforming the processed signal data to a frequency domain;
extracting a DC offset component from the transformed signal data; and
using unextracted data to predict a blood analyte condition.

9. The method of claim 8, wherein the blood analyte condition comprises a concentration of the blood analyte.

10. The method of claim 8, wherein the blood analyte condition comprises a trend over time associated with the blood analyte.

11. The method of claim 8, wherein the blood analyte comprises glucose.

12. The method of claim 8, further comprising adjusting signal data to reduce scale discrepancies.

13. The method of claim 8, further comprising processing the signal data to compute discrepancy measure.

14. The method of claim 8, further comprising adjusting the offset to reduce discrepancy.

15. A method for estimating a blood analyte condition using data from a non-invasive blood monitor, the method comprising the steps of:
receiving signal data from a non-invasive blood monitor, wherein the signal data comprises:
a frequency component corresponding to a heart rate pulse of a user of the non-invasive blood monitor;
a non-pulsatile blood component corresponding to light reflected from within a blood vessel; and
a tissue-dependent DC offset component;
extracting the non-pulsatile blood component from the signal data; and
using the non-pulsatile blood component to estimate a blood analyte condition.

16. The method of claim 15, further comprising:
extracting the frequency component; and
using the frequency component with the non-pulsatile blood component to estimate the blood analyte condition.

17. The method of claim 15, wherein the blood analyte condition comprises a trend over time in a concentration of the blood analyte.

18. The method of claim 15, wherein the blood analyte condition comprises a concentration of the blood analyte.

19. The method of claim 15, wherein the blood analyte comprises blood glucose.

20. The method of claim 15, wherein the non-invasive blood monitor comprises a pulse oximeter, and wherein the blood analyte comprises oxygen.
